(19) United States
(12) Patent Application Publication
(10) Pub. No.: US 2009/0132029 A1
(43) Pub. Date: May 21, 2009

(54) Combination of a vascular prosthesis and retaining element

(76) Inventor: Helmut Zuhlke, Lutherstadt Wittenberg (DE)

Correspondence Address: REED SMITH LLP, Suite 1400, 3110 Fairview Park Drive, Falls Church, VA, 22042, US

(21) Appl. No.: 12/320,190

(22) Filed: Jan. 21, 2009

Related U.S. Application Data
(62) Division of application No. 10/470,989, filed on Apr. 8, 2004.

(30) Foreign Application Priority Data
Feb. 1, 2001 (DE) 101 04 361.8

(51) Int. Cl. A61F 2/06; A61B 19/00

(52) U.S. Cl. 623/1.36; 128/898; 623/1.11

(57) ABSTRACT

A combination of a vascular prosthesis and retaining element, whereby a tubular retaining element (2) is arranged on at least one end of the vascular prosthesis (1), such that the above at least partly extends into the vascular prosthesis (1).

### CROSS REFERENCE TO RELATED APPLICATIONS

This application is a Divisional of U.S. application Ser. No. 10/470,989 filed Apr. 8, 2004, which is a National Stage of PCT/EP2002/00941 filed Jan. 30, 2002. Priority is claimed based on U.S. application Ser. No. 10/470,989 filed Apr. 8, 2004, which claims the priority of PCT/EP2002/00941 filed Jan. 30, 2002, which claims the priority of German Application 101 04 361.8 filed Feb. 1, 2001, all of which is incorporated with reference.

### FIELD OF THE INVENTION

The invention relates to a combination of a vascular prosthesis and a retaining element said combination being in particular employed for the treatment of chronic occlusion illnesses.

### BACKGROUND OF THE INVENTION

The bypassing methods traditionally used for the treatment of occlusive arterial disease incorporating artificial or body's own vascular substitutes entail a major risk of infection on account of the high degree of invasiveness involved. As an alternative to the bypass method the so-called disobliteration plasty is frequently employed which provides for the vessel to be opened proximally to the area to be treated, with the obliteration cylinder to be removed being isolated and cut using a fine spatula-like instrument. Over the distally extending intimal cylinder a ring stripper is mounted which by exerting slight pressure is circularly advanced peripherally with the intimal dissectate being tensioned simultaneously. At the end of the stenotic area the dissectate is cut through with the help of a rope being arranged inside the ring stripper. Although compared with the bypass method the infection risk is lower in this case, another intimal cylinder (intimal hyperplasia) may often form within the disobliterated vessel which in the long run will again require an operative intervention.

Another possibility for the treatment of stenotic areas is to use stents (placeholders, vascular braces). These systems are made of a variety of materials and introduced into the stenotic vessel area where they are dilated by exerting great pressure with the aim to keep the vessel permanently open. This, however, has a drawback in that the deformed intimal cylinder will remain within the vessel system. As a result of the severe mechanical stresses arising when introducing and expanding the stents hyperplasia of the intima induced by dilatation or injuries may frequently occur in the immediately adjacent areas.

For that reason, there is a need for appliances that allow the treatment of occlusive arterial diseases without suffering the disadvantages associated with the state of the art.

### BRIEF DESCRIPTION OF THE INVENTION

According to the invention this objective is reached by a combination of a vascular prosthesis and a tubular retaining element whereby a retaining element is arranged on at least one end of the vascular prosthesis in such a way that said element projects into the vascular prosthesis at least partially.

The combination according to the invention is guided to the usage site where the tubular retaining element after dilatation has taken place braces the vascular prosthesis intraluminally within the vessel to be treated. Basically, the combination according to the invention may be put to use in any desired body area where a surgical placement can be performed. Preferably, it will be employed in the treatment of chronic occlusive arterial diseases of the large pathways, in particular in the extremities and the pelvis planes.

As tubular retaining element a retaining element is preferably used whose buildup and material is at least similar to that used for vascular supporting elements (stents, placeholders). In this manner, the retaining element may be arranged at least partially in the wall of the vascular prosthesis with the retaining element being preferably located in such a way that it projects at least partially into the inner lumen of the vascular prosthesis. In this preferred embodiment of the invention the bracing or fixation of the vascular prosthesis through the retaining element will be particularly firm. Moreover, the combination of this embodiment according to the invention can simply be assembled using individual components (retaining element and vascular prosthesis).

As internal retaining element, the element may completely be arranged within the vascular prosthesis. This embodiment provides for the combination according to the invention to incorporate another retaining element (an external element) preferably arranged on the outside of the vascular prosthesis so that a seamless transition between vascular prosthesis and the inner wall of the vessel to be treated is created.

As per another advantageous embodiment the retaining element is arranged in such a manner that it projects only partially into the vascular prosthesis and extends partially (i.e. beyond the distal end of the vascular prosthesis) out of the vascular prosthesis. In this especially preferred embodiment a single retaining element will be sufficient to enable both the bracing of the vascular prosthesis within the vessel under treatment and the uniform transition between vascular prosthesis and inner wall to be achieved.

Particularly preferred is a combination which provides for the retaining element to be arranged within the prosthesis by more than 50% of its length and outside of it by less than 50% of its length which brings about an excellent bracing of the vascular prosthesis.

Basically, one or several retaining elements may be located on each end of the vascular prosthesis. In the event retaining elements are arranged on both ends of the vascular prosthesis in order to secure the vascular prosthesis, the combination can be braced by endoscopic methods within the vessel under treatment. In this embodiment the vascular prosthesis expediently consists of a vascular endoprosthesis, with the combination according to the invention being preferably introduced after the disobliteration of a hyperplastic intimal cylinder to prevent the intimal cylinder from forming again in the disobliterated area of the vessel and enable the remaining intimal steps located proximally and distally of the disobliterated area to be braced by the retaining elements and at the same time fix the vascular endoprosthesis intravascularly.

In accordance with another preferred embodiment one or several retaining elements are arranged on one end of the vascular prosthesis only. In this embodiment the combination according to the invention is moved endoscopically in distal direction to the remaining intimal step by means of a catheter. After expansion of the retaining element the catheter is retracted. The expansion in this case may be effected through the catheter (by means of a balloon catheter, for example). The use of self-expanding retaining elements may as well be expedient, said elements being introduced in compressed form and expanding automatically as soon as an external force (e.g. exerted by the catheter or the vessel) is eliminated. On the proximal end the vascular prosthesis is attached by conventional sewing methods to the vessel under treatment. The benefit derived from this embodiment is that the vascular prosthesis can be most firmly secured and there is only a minimum degree of invasiveness so that the infection risk is brought down. As regards the fluid flow the combination according to the invention is thus located in such a way that the retaining element is arranged distally in the direction of the blood stream.

Furthermore, the firm connection of the vascular prosthesis with one or several retaining elements is also expedient and makes sure these cannot slip at the time of placement or afterwards. This may be achieved, for example, by bonding, soldering, welding or other techniques known to those skilled in the art.

As per an especially advantageous embodiment the vascular prosthesis is an endoluminal vascular prosthesis. In this embodiment the combination according to the invention is suited for the treatment of chronic occlusive diseases with the aid of extra/intravascular hybrid techniques, with the retaining element serving to the brace the vascular endoprosthesis distally, simultaneously dilatating the vessel and securing the intimal step. The vascular endoprosthesis serves to prevent the reformation and hyperproliferation of an intima in places where the intimal dissectate has been obliterated.

The vascular prosthesis in this case preferably consists of PTFE (polytetrafluoroethylene) or double Dacron velour or other materials that enable the formation of as thin-walled as possible vascular grafts due to their good stability and elasticity properties. The vascular prosthesis here has a diameter that is matched to the lumen diameter of the respective vessel. It may have a uniform transverse diameter and/or lumen but may as well have a conical form and in this way adapt to an anatomical narrowing of vessels from the proximal to the distal end.

The retaining elements employed are preferably compressible and made of one or several materials that after expansion remain stable and, preferably, possess so-called “shape memory” properties. Aside from plastics, suitable materials are in particular iron, tantalum, titanium, niobium, platinum and as well as alloys consisting of at least one of these metals combined with another metal and, especially, Nitinol. (This material is excellently suited for the application since it lends itself to the manufacture of self-expanding retaining elements having shape memory properties.) Furthermore, the retaining elements may be provided with biocompatible coatings (especially in areas where they are in contact with the intima of the vessel to be treated).

In accordance with a particularly expedient embodiment stents are employed as retaining elements. In this embodiment the combination according to the invention can be assembled in a technically simple way using different components (prosthesis and stents) each of which being commercially available. In this case stent types may, for example, be employed that are expanded by the application of a pressure acting from within its lumen (e.g. through the action of a balloon catheter). Self-expanding stents may also be appropriately put to use.

An especially preferred embodiment provides for a retaining element (preferably a stent) that is secured on one end of the vascular prosthesis (preferably a vascular endoprosthesis) in such a manner that it partly (preferably by more than 50% of its length) projects into the inner lumen and partly (preferably by less than 50% of its length) extends out of it in distal direction.

The invention is described in more detail by way of a FIGURE and an embodiment as outlined below.

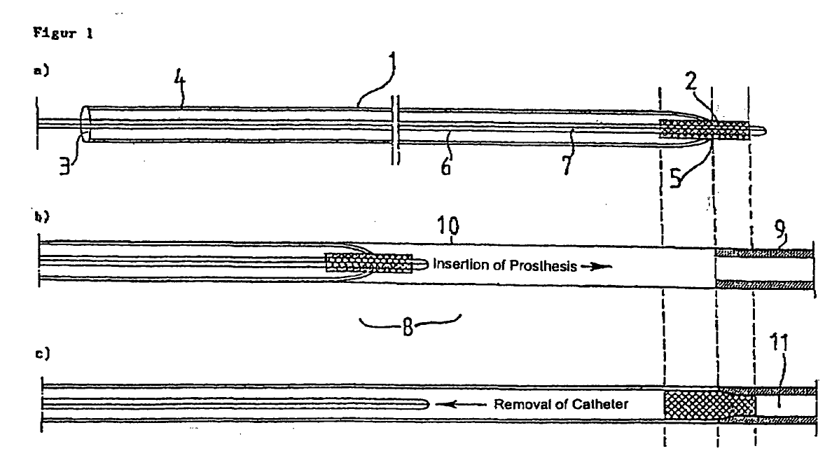

FIG. 1 (a, b, c) shows a longitudinal section through a combination consisting of a vascular endoprosthesis and a stent of which a side view is shown.

FIG. 1 (a, b, c) shows a longitudinal section through a combination consisting of a vascular endoprosthesis and a stent of which a side view is shown.

The combination illustrated in the FIGURE comprises a vascular endoprosthesis 1 on which end a retaining element in the form of a stent 2 has been arranged in such a way that it is situated in the inner lumen 3 of the prosthesis tube 4 by more than half of its length and projects out of the distal end 5 of the vascular endoprosthesis 1 by less than half of its length. The stent 2 is a highly flexible and bendable element made of Nitinol. The total length of the vascular endoprosthesis amounts to approx. 40 cm.

As shown in FIG. 1b the combination is advanced endoscopically within the partially disobliterated (i.e. peeled off in parts) vessel 8 up to the remaining intimal step 9 with the help of a placement catheter 6 (having an inner lumen 7 capable of accommodating a mandrin or flushing solution). The intimal step 9 (intimal lip) is formed by the remaining intima of the blood vessel 8 adjacent to the disobliterated area. The placement catheter 6 has been designed as a balloon catheter onto which the stent 2 is mounted from the outside. When the intimal step has been reached the combination is arranged in such a way that the vascular endoprosthesis 1 is situated proximally adjacent to the intimal step 9. By dilatating the balloon catheter 6 the stent 2 is radially expanded and in this way braces the vascular endoprosthesis 1 against the remaining adventitia 10 of the disobliterated vessel 8 while simultaneously creating a uniform transition between the inner lumen of the vascular endoprosthesis 1 and the inner lumen of the vessel 11 under treatment.

Subsequently, the balloon catheter 6 is re-compressed and removed from the vessel in proximal direction. Remaining in the vessel are the combination of the vascular endoprosthesis 1 and now radially expanded stent 2 with the prosthesis tube 4 intraluminally contacting the adventitia thus preventing an intima from forming anew. The tight bracing of the distal end 5 of the vascular endoprosthesis 1 brought about by the radially expanded stent 2 within the treated vessel at the same time prevents muscle cells of the remaining intimal step 9 from growing onto the adventitia 10 of the disobliterated area of the vessel 8 under treatment.

### EXAMPLE 1

Extra-endoluminal implantation of a combination comprising vascular endoprosthesis and stent to treat a chronic occlusive arterial disease (paVK) in the area of arteria femoralis superficialis.

Having exposed the vessels in the area of the groin (a. femoralis communis, a. profunda femoris and a. femoralis superficialis) the vessel system above the branching off a. profunda femoris is opened and the cut extended into the a. femoralis superficialis (artery connecting to the lower leg).

Closely behind the branching off a. femoralis superficialis the intimal cylinder is peeled off, cleared circularly, cut through and disobliterated by means of a ring stripper up to the level of a. poplitea. (Preparation of the adductor canal, also termed receiving segment, since the collateral vessels of the a. profunda femoris are found here.) This operation is carried out under radiographic observance.

Having removed the intimal cylinder the open lumen of the a. poplitea is examined by probe and a guide wire introduced in the a. poplitea into the 2^{nd} and 3^{rd} segment. Making use of the guide wire a thicker guide wire is then placed in position. Having again checked the situation by radiography the prosthesis device consisting of vascular endoprosthesis and stent is then introduced. Using a balloon catheter onto which the folded up stent is placed the prosthesis device is moved forward in distal direction. The portion of the stent outwardly projecting from the vascular endoprosthesis is pushed over the intimal lip (remaining muscle layer), and subsequently the entire stent is expanded with the help of the balloon. Under radiographic observance the stent together with the vascular endoprosthesis is unfolded with the aid of the balloon in the area of the transition of the disobliterated a. femoralis superficialis and a. poplitea and, following this, the catheter is retracted causing the prosthesis to be stripped off the catheter. Afterwards, the catheter is pulled up towards the groin region. As a result of the intraluminal stent the vascular endoprosthesis is fixed within the vessel. Moreover, the vessel itself is slightly dilatated in this location. Simultaneously, the intimal step is also fixed by the stents.

Following the intravascular fixation of the distal end of the vascular endoprosthesis by the dilatation of the stent, for which purpose an as thin-walled prosthesis (either of PTFE or double Dacron velour) can be employed, the catheter is retracted. Over its entire length the vascular endoprosthesis may have a uniform lumen of a diameter ranging between 6 and 8 mm; however, a tapering prosthesis enlarging from 6 to 8 mm may also be used.

As far as arteriotomy is concerned, the prosthesis is cut to size in the groin region and connected to the a. femoralis superficialis using a conventional sewing technique, whereby a transluminal technique must be employed for the posterior wall while the anterior wall can be connected to the opened a. femoralis communis by sewing employing a typical technique. Additionally, an extension plasty of the a. femoralis communis may be provided. Having completed the anastomoses the flow of blood to the periphery is released. This method offers the advantages of minor invasiveness (only one vessel exposure)—which minimizes the infection rate and orthotopic vessel reconstruction—, no further traumatization for an implant depot, and time savings. The intraluminal prosthesis in this case entails a lower risk of hyperplasia than an extra-anatomically placed prosthesis. In the pelvis planes the technique may also be employed retrogradedly, i.e. from the groin side after a disobliteration graft of the a. iliaca extema and a. iliaca communis.

1. A method for treatment of chronic occlusive arterial diseases, comprising the steps of:
opening a vessel proximally to an area to be treated;
peeling off and disobliterating an intimal cylinder;
with a catheter, introducing combination of a vascular endoprosthesis and a tubular retaining element located at a distal end of the vascular endoprosthesis, wherein the tubular retaining element is coupled with the vascular endoprosthesis only at a distal end of the vascular endoprosthesis and no tubular retaining element is coupled with the vascular endoprosthesis at a proximal end of the vascular endoprosthesis, the tubular retaining element projecting at least partially into the vascular endoprosthesis, and the proximal end of the endoprosthesis is configured to be sewed to the vessel to be treated,
pushing the retaining element outwardly so as to project outwardly from the vascular endoprosthesis over a remaining muscle layer;
expanding the retaining element;
retracting the catheter; and
sewing the proximal end of the vascular endoprosthesis to the vessel.

2. The method according to claim 1, wherein the tubular retaining element projects at least partially into the inner lumen of the vascular endoprosthesis.

3. The method according to claim 1, wherein the tubular retaining element projects distally out of the vascular endoprosthesis.

4. The method according to claim 3, wherein the tubular retaining element is arranged by more than 50% of its length within the vascular endoprosthesis.

5. The method according to claim 1, the vascular endoprosthesis is an endoluminal vascular prosthesis.

6. The method according to claim 1, wherein the vascular endoprosthesis is firmly connected with the tubular retaining element.

7. The method according to claim 1, wherein the vascular endoprosthesis is made of PTFE or double Dacron velour.

8. The method according to claim 1, wherein the vascular endoprosthesis has a tapering form.

9. The method according to claim 1, wherein the tubular retaining element is compressible and radially expandable.

10. The method according to claim 1, wherein the tubular retaining element comprises at least one of at least one plastic material, at least one metal selected from the group consisting of iron, tantalum, titanium, niobium, and platinum and an alloy, the alloy consisting of at least one metal selected from the group consisting of iron, tantalum, titanium, niobium, and platinum combined with at least another metal.

11. The method according to claim 1, wherein at least a part of the tubular retaining elements is a stent.

12. The method according to claim 10, wherein the another metal is Nitinol.
